(19) United States
(12) Patent Application Publication
(10) Pub. No.: US 2023/0309912 A1
(43) Pub. Date: Oct. 5, 2023

(54) PHONE CASE WITH SLIDABLE LENSES AND RELATED APPLICATION FOR IDENTIFYING SKIN CONDITIONS

(71) Applicant: L'Oreal, Paris (FR)

(72) Inventors: Brady Rowe, Clark, NJ (US); Grace Meng, Clark, NJ (US)

(73) Assignee: L'Oreal, Paris (FR)

(21) Appl. No.: 17/710,136

(22) Filed: Mar. 31, 2022

(51) Int. Cl. A61B 5/00; G16H 50/30; G16H 30/40; G06T 7/00

(52) U.S. Cl. CPC A61B 5/441; G16H 50/30; G16H 30/40; G06T 7/0012; G06T 2207/30088

(57) ABSTRACT

A method of detecting a skin condition, the method including placing a communication device into a case, sliding a first lens, a second lens, or both lenses along a support member on the case to cover a camera of the communication device, taking an image with the camera of the communication device, using image processing with an application on the communication device to identify one or more skin features, and recommending a treatment for the one or more skin features.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a method of detecting a skin condition including placing a communication device into a case, sliding a first lens, a second lens, or both lenses along a support member on the case to cover a camera of the communication device, taking an image with the camera of the communication device, and detecting a skin condition with an application on the communication device is disclosed.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an example system for detecting a skin condition, in accordance with the present technology;

FIG. 2 is another system for detecting a skin condition, in accordance with the present technology;

FIG. 3A is another system for detecting a skin condition, in accordance with the present technology;

FIG. 3B is an example system for detecting a skin condition in operation, in accordance with the present technology;

FIG. 4 is a flowchart of a method for detecting a skin condition, in accordance with the present technology; and

FIG. 5 is an example system for detecting a skin condition in operation, in accordance with the present technology.

### DETAILED DESCRIPTION

While illustrative embodiments have been illustrated and described, it will be appreciated that various changes can be made therein without departing from the spirit and scope of the invention.

Disclosed is a system for detecting a skin condition including a case, a communication device, and an application (“app”) on the communication device. The app is configured to work in conjunction with a case that includes one or more lenses configured to modify an image taken with the optical system (camera) of the communication device. In operation, a user may take an image (photo) of their skin with a communication device, with the one or more lenses covering the optical system of the communication device. In some embodiments, the app uses the modified image, image processing techniques, and/or AI to analyze the user's skin to identify skin conditions including, by not limited to, overall skin condition, acne, wrinkles, fine lines, scars, wounds, tumors, fungal infections, bacterial infections, viral infections, and dryness. In some embodiments, the app also assesses environmental conditions that can influence the state of the user's skin. Example conditions include, but are not limited to UV index, air quality, pollen, weather, temperature, and humidity. In some embodiments the app stores the image and the detected skin condition as a user's personal skin history. In some embodiments, the app also takes into account the user's personal skin history when detecting the skin condition. In some embodiments, the app includes a cloud platform. In some embodiments, the cloud platform can aggregate the images as a user's personal skin history.

In some embodiments, the app also assesses user inputs, including current skin condition, past skin conditions, past diagnoses (which can be both user inputs and past app diagnoses), and specific skin concerns. In some embodiments, the user can select specific skin concerns or conditions. In some embodiments, the user selection may include a questionnaire, giving the user a set of prompts to respond to in order to determine what skin condition they may have or what treatment may work best for treating said skin condition. In some embodiments, the questionnaire may help determine which of the lenses to slide over the camera when taking an image to detect the skin condition of interest.

In some embodiments, the app detects a skin condition. In some embodiments, the app detects a skin condition, and recommends a treatment. In some embodiments, the treatment is a cosmetic, a skin care product, a topical ointment or cream, an antifungal, an antibacterial treatment, an anti-viral treatment, and/or a recommendation to speak with a medical professional.

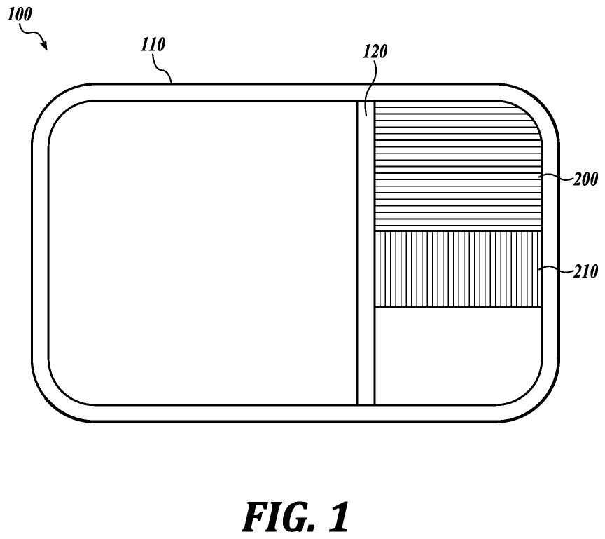

FIG. 1 is an example system for detecting a skin condition, in accordance with the present technology. In some embodiments, the system 100 includes a case 110, a support member 120, a first lens 200, and a second lens 210.

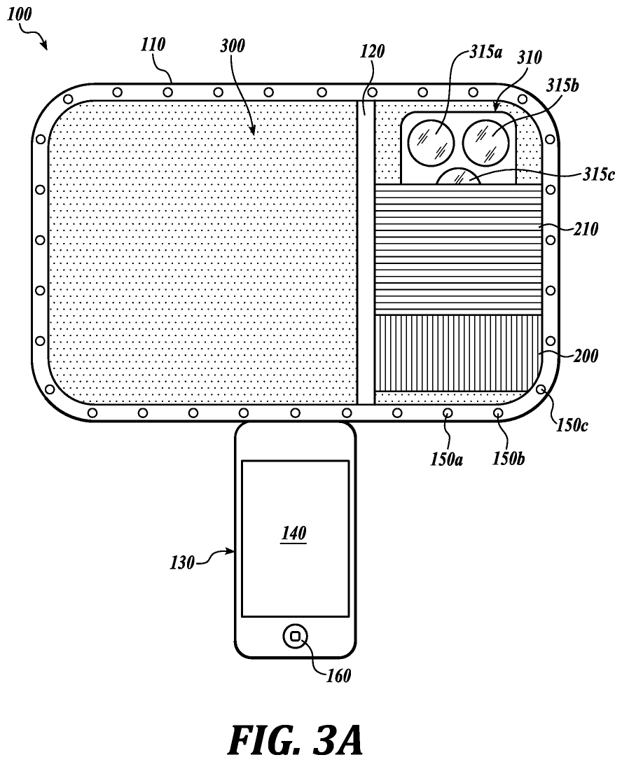

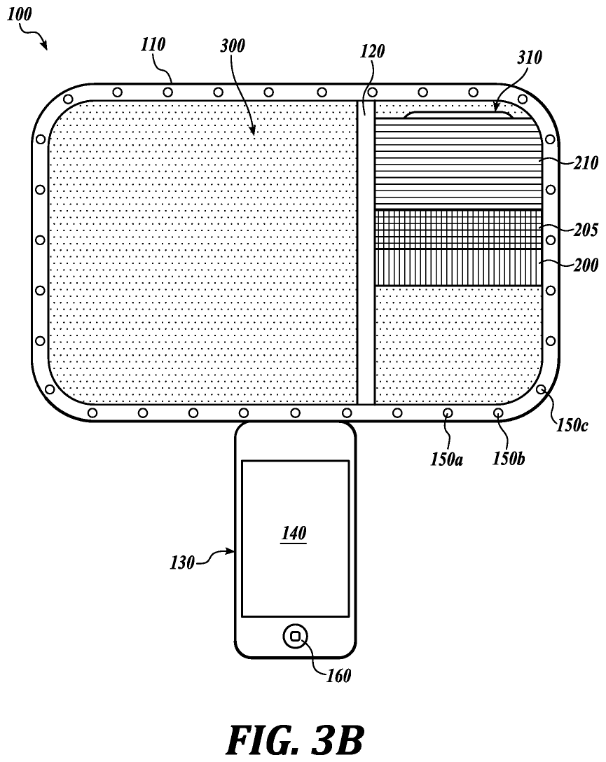

In some embodiments, the case is configured to hold a communication device (as shown in FIG. 3A-3B). In some embodiments, the case 110 is shaped to fit the communication device exactly, but in some embodiments, the case 110 may be thicker, or shaped in a way that extends the size or changes the shape of the communication device. In some embodiments, the case 110 is made of a flexible material, but in other embodiments, the case 110 may be rigid.

In some embodiments, the first lens 200 and the second lens 210 are slidably coupled to the case 110, so that the lenses 200, 210 can be slid along the length of the support member 120. In some embodiments, the case 110 includes a groove on a portion of the case 110 that holds the first lens 200 and the second lens 210. In some embodiments, the case 110 may have two grooves, so that the first lens 200 and the second lens 210 each have a separate groove. In operation, the lenses 200, 210 are slotted into the groove, disposed between the edge of the case 110 and the support member 120 so that they slide along the length of the support member 120. In some embodiments, the case is manufactured to that the lenses 200, 210 are already coupled to the case 110. In some embodiments, the lenses 200, 210 may be separate from the case 110, and can be inserted into the case by a user.

In some embodiments, the case 110 includes a support member 120. In some embodiments, the support member 120 extends along the entire width of the case 110, as shown in FIG. 1. In some embodiments, the support member 120 extends along the length of the case 110 horizontally. In some embodiments, the support member 120 has a groove to hold the first lens 200 and the second lens 210. In some embodiments, the support member 120 may have two grooves, so that the first lens 200 and the second lens 210 each have a separate groove. In operation, the lenses 200, 210 can be slid along the groove on the support member and the groove on the case to cover a camera of the communication device (as shown in FIG. 3B).

In some embodiments, the case 110 includes at least a first lens 200 and a second lens 210. While only two lenses 200, 210 are illustrated, any number of lenses can be incorporated onto the case 110. The first lens 200 and the second lens 210 may be any type of lens, including a parallel lens, a polarizer, a cross-polarizer, a linear polarizer, a circular polarizer, an elliptical polarizer, a colored lens, a magnification lens, and the like. In some embodiments, the first lens 200 is a parallel lens in a first orientation. In some embodiments, the second lens 210 is a parallel lens in a second orientation, perpendicular to the first orientation. In some embodiments, the first lens 200 is a vertical linear polarizer, and the second lens 210 is a horizontal linear polarizer. In operation, when the first lens 200 and the second lens 210 are layered on top of one another, the image is cross polarized. The first lens 200 and the second lens 210 may be any combination of lenses, for example, the first lens 200 may be a circular polarizer, and the second lens 210 may be another type of lens, such as a magnification lens. As such, in this example, when the first and second lens are layered on top of one another, the image is polarized and magnified. In some embodiments, the second lens 210 is configured to slide underneath the first lens 200.

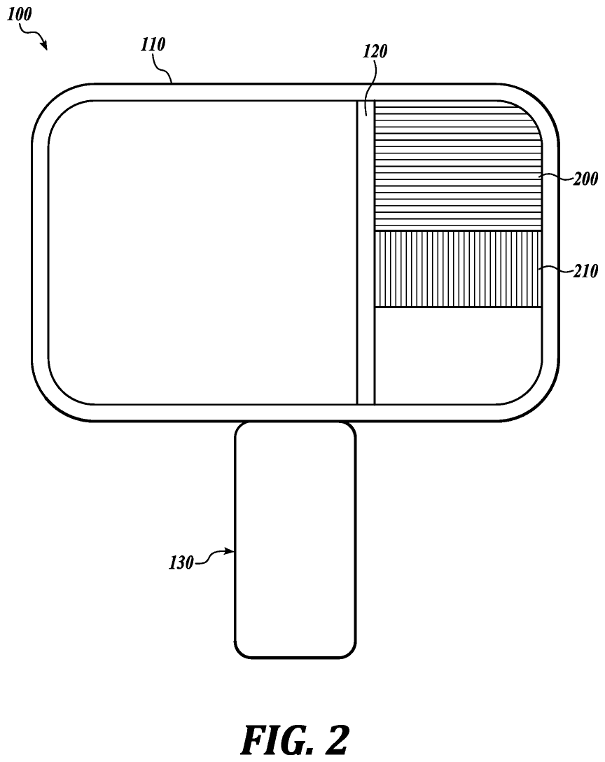

FIG. 2 is another system for detecting a skin condition, in accordance with the present technology. In some embodiments, the system 100 further includes a handle 130. While the handle 130 is illustrated similarly to a mirror handle in FIG. 2, the handle 130 may take any form, including a long support handle used to hold the camera at a distance from a user.

In operation, a user can hold the system 100 with the handle 130, and slide the first lens 200, the second lens 210, or both lenses 200, 210 over the camera of a communication device, and take an image of the skin, such as a face, arm, neck, or chest. In some embodiments, the user can take an image of a skin condition, such as acne, a wart, a scar, a wound or a raised bump on the skin.

FIG. 3A is another system 100 for detecting a skin condition, in accordance with the present technology. In some embodiments, the system 100 includes a power source 140, inside the handle 130, a plurality of lights 150a, 150b, 150c, along the edge of the case 110, and one or more actuators 160.

In some embodiments, the plurality of lights 150a, 150b, 150c are LED lights. In some embodiments, the plurality of lights 150a, 150b, 150c are UVA LED lights. Although the plurality of lights 150a, 150b, 150c are illustrated as being disposed around the entirety of case 110, in some embodiments, the plurality of lights 150a, 150b, 150c may be disposed only partially along the edge of the case, such as along only the top edge of the case, or only the top and bottom edge of the case. In some embodiments, a power source 140 is communicatively coupled to the plurality of lights 150a, 150b, 150c. In some embodiments, such as those illustrated in FIGS. 3A-3B, the power source 140 is located on or inside the handle 130, but in other embodiments, the power source 140 is on or inside the case 310 or may be inside the communication device 300 itself. In some embodiments, the power source 140 is communicatively coupled the actuator 160. The power source 140 may be a battery but can take the form of any suitable power source. In some embodiments, the handle 130 of the system 100 includes an actuator 160.

In some embodiments, the actuator 160 is a button, as illustrated in FIGS. 3A-3B, but in some embodiments, the actuator 160 is a switch, a capacitive touch-type button, a lever, or the like. In some embodiments, the actuator 160 is communicatively coupled to the communication device 300.

In operation, a user may actuate the actuator 160 to turn on the plurality of lights 150a, 150b, 150c, take an image with the communication device 300, or both. In some embodiments, a user may actuate the actuator a first time to turn on the plurality of lights 150a, 150b, 150c, and then actuate the actuator a second time to take the image with the communication device 300. In some embodiments, the user may actuate the actuator a first time to turn on the lights 150a, 150b, 150c, and then actuate the actuator a second time to turn off the lights 150a, 150b, 150c. While the actuator 160 is illustrated as on the handle 130 of the system 100, in some embodiments, the actuator 160 may be on the case 110 or on the communication device 300 itself.

In some embodiments, the system 100 further includes a communication device 300. In some embodiments, the communication device includes an optical system 310 including a plurality of cameras 315a, 315b, 315c. The communication device 300 is illustrated as a smartphone in FIGS. 3A and 3B, but the communication device may take any number of forms, including a tablet, laptop screen, desktop monitor, smart watch, and the like.

While the optical system 310 is illustrated as having three cameras 315a, 315b, and 315c, the communication device 300 may have any number of cameras. Further, while the optical system 310 is illustrated as being square and disposed on a right-hand side of the back of the communication device 300, the optical system 310 may take any shape or configuration. For example, the optical system 310 may be on the front of the communication device 300, i.e., on the same side as a screen of the communication device 300, so that the user may visualize the image before taking it with the system 100. In some embodiments, the optical system 310 is a single camera 315.

FIG. 3B is an example system 100 for detecting a skin condition, in operation, in accordance with the present technology. In some embodiments, the first lens 200, the second lens 210, or an overlap 205 of both lenses cover the optical system 310 before an image is taken.

In some embodiments, the first lens 200 and the second lens 210 are configured to modify the image taken by the optical system 310 of the communication device 300. In operation, the first lens 200, the second lens 210, or the overlap 205 of both lenses are slid over optical system 310 of the communication device 300. In some embodiments, the first lens 200 modifies the image in a first way. In some embodiments, the second lens 210 modifies the image in a second way. In some embodiments, the overlap 205 of the lenses 200, 210 modifies the image in a third, distinct way. In some embodiments, there may be more than two lenses. In some embodiments, the overlap 205 of the two or more lenses provide even more image modifications. In some embodiments, the lenses 200, 210 are the same size. While the lenses 200, 210 are shown to overlap 205 only partially in FIG. 3B, the lenses 200, 210 may completely overlap over the optical system 310 of the communication device, so that all the cameras 315a, 315b, 315c of the optical system 310 are covered by both lenses 200, 210.

The first lens 200 and the second lens 210 may be any type of lens, including a parallel lens, a polarizer, a cross-polarizer, a colored lens, a magnification lens, and the like. In some embodiments, the first lens 200 is a parallel lens in a first orientation. In some embodiments, the second lens 210 is a parallel lens in a second orientation, perpendicular to the first orientation. In some embodiments, the first lens 200 is a vertical linear polarizer, and the second lens 210 is a horizontal linear polarizer. In operation, when the first lens 200 and the second lens 210 are layered on top of one another, the image is cross polarized.

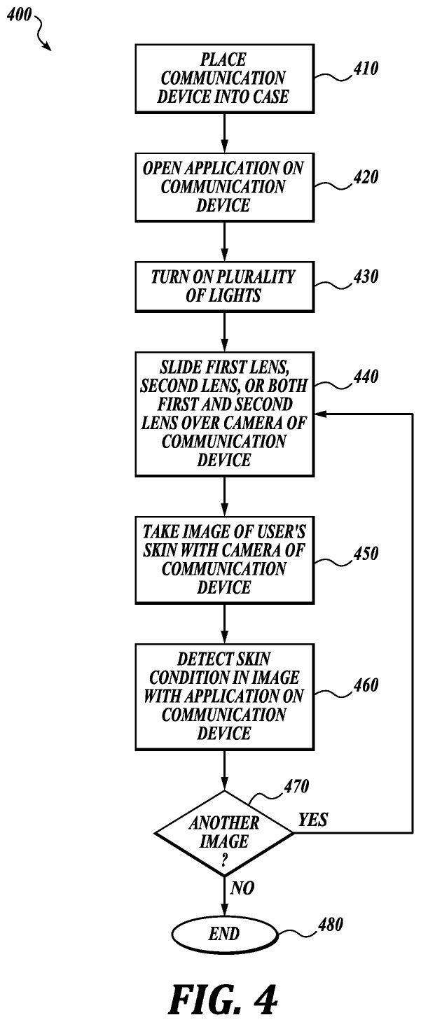

FIG. 4 is a flowchart representing an example method for detecting a skin condition, in accordance with the present technology.

In step 410, a user places a communication device into a case, such as case 110. In some embodiments, the communication device may be slid into the case. In some embodiments, the case is flexible, so the case may be stretched to accommodate the communication device. In some embodiments, the case is snapped onto the communication device.

Optionally, in step 420, the user can open an application (app) on the communication device. In some embodiments, step 420 occurs before step 410. In some embodiments, the app does not need to take the photo to analyze it, i.e., the photo may be uploaded into the app from another, separate application, such as a general camera application.

Optionally, in step 430, a plurality of lights (such as plurality of lights 150a, 150b, 150c in FIGS. 3A-3B) are turned on. In some embodiments, the plurality of lights are LED lights, or UVA LED lights. In some embodiments, the plurality of lights is located on the case. In some embodiments, the plurality of lights may be a separate component, not attached to the case. In some embodiments, the user can take the image without turning on the plurality of lights.

In step 440, the first lens, the second lens, or both lenses are slid over a camera of the communication device. In some embodiments, the first lens, second lens, or both lenses may be slid over more than one camera in an optical system, as shown in FIGS. 3A-3B. In some embodiments, the first lens modifies the image taken in step 450 in a first way. The second lens may modify the image taken in step 450 in a second way, and the overlapped first and second lens may modify the image in a third way.

In step 450, an image of the user's skin is taken with the camera of the communication device. In some embodiments, the image is of the user's face, but in other embodiments, the image can be any image of skin, including but not limited to an arm, a neck, a leg, a stomach, a shoulder, a back, or a skin condition of interest (such as a wrinkle, fine line, acne, blemish, lump, or infection). In some embodiments, the image may be one or more photos of the user's skin. In some embodiments, the image may be a 360-degree scan of the user's skin.

In step 460, an app on the communication device detects a skin condition in the image of the user's skin. In some embodiments, as described herein, the app uses the image modified by the one or more lenses, image processing, and/or AI to analyze the user's skin. In some embodiments, the app is configured to identify skin conditions including by not limited to overall skin condition, acne, wrinkles, fine lines, scars, wounds, tumors, fungal infections, bacterial infections, viral infections, and wounds. In some embodiments, the app also assesses environmental conditions that can influence the state of the user's skin. Example environmental conditions include, but are not limited to UV index, air quality, pollen, weather, temperature, and humidity. In some embodiments the app stores the image as a user's personal skin history. In some embodiments, the app also takes into account the user's personal skin history. In some embodiments, the app also assesses user inputs, including current skin condition, past skin conditions, past diagnoses (user inputs and/or past app diagnoses), and specific skin concerns. In some embodiments, the app includes a questionnaire to assess user inputs. In some embodiments, the user can select specific skin concerns or conditions from the questionnaire. In some embodiments, the questionnaire may include giving the user a set of prompts to respond to in order to determine what products would work best for their specific needs. In some embodiments, the app can alert the user to use a different lens (or combination of lenses) to better visualize the user's skin based on the responses to the questionnaire.

In some embodiments, the app detects a skin condition. In some embodiments, the app detects a skin condition, and recommends a treatment. In some embodiments, the treatment is a cosmetic, a skin care product, a topical ointment or cream, an antifungal, an antibacterial treatment, and anti-viral treatment, and/or a recommendation to speak with a medical professional.

In step 470, the user can take another image. In some embodiments, the user can take another image of the same portion of skin with a new lens, a new portion of skin, or an image with the lens configuration recommended by the app. If the user decides to take another image, the method 400 proceeds back to block 440. If the user decides to not take another image, the method 400 proceeds to block 480.

In block 480, the method ends.

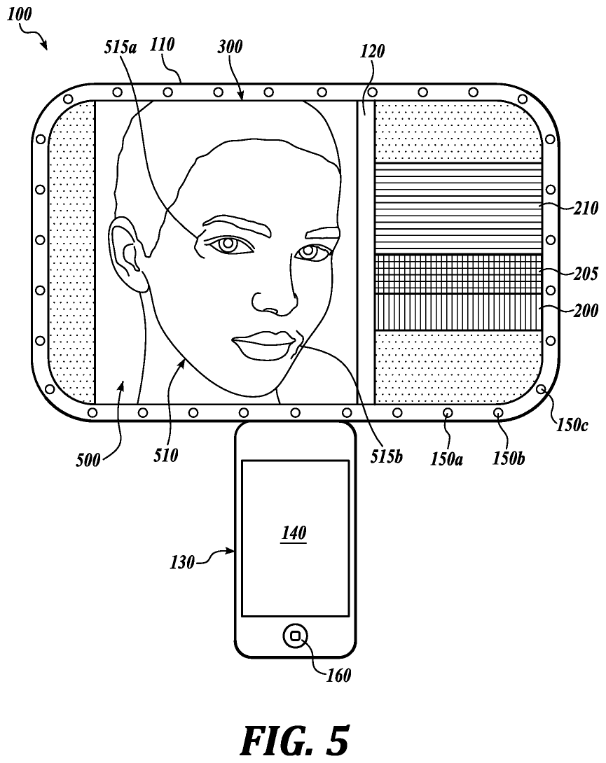

FIG. 5 is an example system for detecting a skin condition in operation, in accordance with the present technology. In some embodiments, the system 100 includes a case 110 that is configured to hold a communication device 300 so that the screen 500 of the communication device 300 is accessible and visible to a user. In these embodiments, the user can see themselves as they take an image with the communication device 300, when the one or more lenses 200, 210 are overlapped over the camera of the communication device 300.

In operation, a user may visualize their skin 500, such as their face, as shown in FIG. 5 on the screen 500 of the communication device 300. In some embodiments, the user may visualize one or more skin features 515A, 515B on their skin 500 on the screen 500 of the communication device 300.

In some embodiments, the plurality of lights 150a, 150b, 150c and the one or more lenses 200, 210 are configured to take photos with the camera of the communication device 300. In some embodiments, the communication device 300 can determine if the light conditions are suitable for detecting the one or more features of the user's skin. In some embodiments, the communication device 300 can sense as to whether the user is in adequate lighting conditions to detect a feature on the user's skin 500 with the lenses 200, 210. If it is determined that the user is not in adequate lighting conditions, the plurality of lights 150a, 150b, 150c may illuminate the user's face 500 to better detect the user's skin features 515A, 515B. If this is still not enough light to detect the user's features, such as if the user is in a dark room, the communication device 300 may alert the user to move to another location. After the lighting conditions are determined to be adequate to detect the user's features, the camera may visualize an image of the user's face.

When the user takes an image of their skin, the application of the smart phone may apply image processing techniques to the image. The communication device 300 can then assign a probability to each pixel of each image as being either (1) a feature, (2) a facial landmark, or (3) normal skin based on the color of each pixel. In some embodiments, the feature is a wrinkle, a fine line, pigmentation, hyper pigmentation, or acne. In some embodiments, the feature is detected based on the color of the pixel. In some embodiments, the feature ranges from pink to red. In some embodiments, the feature is any color that is not the color of the user's normal skin. For example, the processor 160 may identify hyper pigmentation on a user's skin by identifying pixels that are pink or red in color. The application can then recommend a variety of treatments based on the skin features detected.

In some embodiments, the communication device 300 detects wrinkles or fine lines on the user's face by detecting pixels that are darker in color than the user's skin tone on the user's face in an unbroken line segment across the user's face. In some embodiments, the application assigns a probability to each pixel as being either a wrinkle or the user's unblemished skin based on color. In some embodiments, the application can then direct the user to apply or purchase either a wrinkle treatment, a concealer, or both, for effectively treating and concealing the wrinkle or fine line.

In some embodiments, the application sets a probability threshold for determining whether or not to recommend a skin formula or treatment to the pixels that have a probability of being a skin feature, such as a wrinkle, fine line, acne, pigmentation, or hyper pigmentation. In some embodiments, the probability threshold is hardcoded into the application 160. In some embodiments, the probability threshold may be adjusted to the user's preferences or needs. For example, the threshold may need to higher if the skin feature and the user's skin tone are not very distinct from each other. The threshold may likewise be lowered when the desired skincare and the undesired skincare are distinct from one another, such as when a user has a lighter skin tone, and the feature is red or pink in color. In some embodiments, the probability threshold is set at 85%, 90%, 95%, or 100%. If a pixel exceeds the probability threshold, the application directs the user to apply skin formula or seek treatment, including treatment from a medical professional. In some embodiments, the application can detect one or more skin features by detecting an unbroken line of pixels on the user's face. In this case, if the average of the pixels within the unbroken segment exceeds the probability threshold, the application can direct the user to apply the skin formula to the entire wrinkle on the user's face.

The communication device 300 is capable of performing wireless communication with the case 110 by way of a wireless communication interface circuitry on the case 110. However, communication device 300 is also capable of having a wired connection to the case 110 by way of a USB interface or other electrical connection on the case 110.

1. A method of detecting a skin condition, the method comprising:
placing a communication device into a case;
sliding a first lens, a second lens, or both lenses along a support member on the case to cover a camera of the communication device;
taking an image with the camera of the communication device;
using image processing with an application on the communication device to identify one or more skin features; and
recommending a treatment for the one or more skin features.

2. The method of claim 1, wherein the method further comprises lighting the user's face with a plurality of LED lights on the case.

3. The method of claim 1, wherein the case is configured to hold the communication device so that a screen of the communication device is visible.

4. The method of claim 1, wherein the one or more skin features is selected from a wrinkle, a fine line, acne, a scar, a tumor, a bacterial infection, a fungal infection, a viral infection, hyperpigmentation, or a wound.

5. The method of claim 1, the method further comprising storing the image and the detected skin one or more skin features to generate historical data of a user's skin.

6. The method of claim 1, the method further comprising informing the detection of the skin feature with the historical data of the user's skin.

7. The method of claim 5, wherein storing the image and the detected one or more skin features comprises storing with a cloud platform on the application.

8. The method of claim 1, wherein the treatment is selected from a cosmetic, a skin care product, a topical ointment or cream, an antifungal treatment, an antibacterial treatment, and anti-viral treatment, or a recommendation to speak with a medical professional

9. The method of claim 1, wherein the first lens is a vertical polarizer.

10. The method of claim 1, wherein the second lens is a horizontal polarizer.

11. The method of claim 1, wherein using image processing comprises using AI to analyze the user's skin.

12. The method of claim 1, wherein using image processing comprises assigning a probability to each pixel of each image as being either a skin feature, a facial landmark, or normal skin based on the color of each pixel.

13. The method of claim 1, wherein using image processing comprises using a segmentation image processing algorithm.

14. The method of claim 1, wherein the method further comprises receiving one or more user input before the user takes the photo.

15. The method of claim 15, wherein the method further comprises selecting one or more skin conditions of concern.

16. The method of claim 15, wherein the method further comprises issuing an alert as to which lens or combination of lenses to slide over the camera before taking the image based on the one or more skin condition of concern.

17. The method of claim 15, wherein the user input comprises a questionnaire.

18. The method of claim 1, wherein the method further comprises considering one or more environmental factors when identifying the one or more skin feature.

19. The method of claim 1, wherein method further comprises:
comparing the one or more skin features with one or more other users; and
using the comparison to inform the treatment recommendation.

20. The method of claim 19, wherein the one or more other users share at least one skin feature.
